(19) United States
(12) Patent Application Publication
(10) Pub. No.: US 2008/0248955 A1
(43) Pub. Date: Oct. 9, 2008

(54) Emulsifiable Concentrates Containing Adjuvants

(76) Inventors: Jeffrey David Fowler, Greensboro, NC (US); Hans Walter Haesslin, Grenzach-Wyhlen (DE); Angelika Haesslin, Grenzach-Wyhlen (DE); Andreas Haesslin, Grenzach-Wyhlen (DE); Michel Haesslin, Grenzach-Wyhlen (DE); Anna Haesslin, Grenzach-Wyhlen (DE); Manfred Vogt, Bad Sackingen (DE); Michelle Weber, Kernersville, NC (US)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/578,735

(22) PCT Filed: Nov. 16, 2004
(86) PCT No.: PCT/US04/38414

§ 371 Date: May 18, 2007

Related U.S. Application Data
(60) Provisional application No. 60/520,561, filed on Nov. 17, 2003.

(51) Int. Cl. A01N 25/32; A01N 43/40; A61K 31/44; A01P 7/00; A01P 13/00; A01P 13/02

(52) U.S. Cl. 504/105; 504/258; 514/345

(57) ABSTRACT

The present invention relates to stable emulsifiable concentrates comprising an oil adjuvant and at least one member selected from the group consisting of herbicidally active 2-[4[(5-chloro-3-fluoropyridin-2-yloxy)-phenoxy]-propionic acid derivatives and quinoline derivative safeners and to the use thereof as a pesticide.

The present invention relates to stable emulsifiable concentrates comprising an oil adjuvant and at least one member selected from the group consisting of herbicidally active 2-[4[(5-chloro-3-fluoropyridin-2-yloxy)-phenoxy]-propionic acid derivatives and quinoline derivative safeners.

The present invention also relates to herbicidal compositions for controlling grasses and weeds in crops of useful plants, especially in crops of maize and cereals, prepared from these emulsifiable concentrates, and to the use of such compositions in controlling weeds in crops of useful plants.

### BACKGROUND OF THE INVENTION

The use of adjuvants for improving the biological activity and/or spray properties of pesticidal compositions is well known. However, certain components, e.g., active ingredients or antidotes, useful in pesticidal formulations are chemically unstable or have limited chemical stability. These stability issues can limit the ability to formulate pesticidal concentrates and restrict the use of additional components, such as adjuvants, in the concentrate formulations.

There is a need for chemically stable formulations containing components having limited chemical stability. Further, it is desirable to have pesticidal concentrates containing “built-in” adjuvants in order to avoid having to add these adjuvants as a tank-mix component.

Emulsifiable concentrate (EC) formulations conventionally contain an active ingredient, one or more surfactants which act as emulsifiers upon dilution of the EC with water and a water immiscible solvent. Typical solvents for conventional EC formulations are aromatic hydrocarbons. These solvents have very low solubilities in water and a high capability of dissolving a wide range of active ingredients.

Due to the presence of the solvent, many pesticides formulated as an EC have advantages such as a higher degree of systemicity and higher overall activity compared to the same pesticide formulated as a wettable powder (WP), water dispersible granule (WG) or suspension concentrate (SC).

The observed efficacy of the combination of ingredients can sometimes be significantly higher than would be expected from the amounts of the individual ingredients used (synergism). The efficacy of the active components can often be improved by addition of other ingredients such as adjuvants.

In view of increasing the ease and safety of handling and dosing of these adjuvants by the end-user and in view of avoiding unnecessary packaging material, it is desirable to develop concentrated formulations which already contain such adjuvants.

U.S. Pat. No. 6,566,308 discloses EC formulations stabilized with a water-miscible polar aprotic solvent.

### SUMMARY OF THE INVENTION

In one embodiment, the present invention relates to an emulsifiable concentrate (EC) comprising

a) a herbicidally effective amount of at least one herbicide;

b) optionally, an amount, which is effective for antagonism of a herbicide, of at least one safener;

c) 5 to 80%, preferably 25 to 70%, by weight of at least one oil adjuvant, wherein said oil adjuvant comprises an oil of vegetable or animal origin, or a mineral oil, alkyl esters thereof or mixtures of those oils and oil derivatives;

d) an amount of at least one water-immiscible solvent sufficient to keep the AI and safener in solution in the presence of the adjuvant; and

e) an emulsifying surfactant system in an amount sufficient to form an oil-in-water emulsion when the formulation is added to water;

with the proviso that a) comprises a herbicidally effective amount of at least one compound of formula I

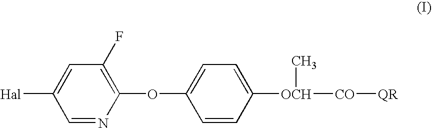

wherein Hal is halogen
Q is oxygen or sulfur,
R is hydrogen, an alkali metal ion, or a quaternary C₁-C₄-alkylammonium group, a C₁-C₆-alkyl group which is straight-chain or branched-chain, and which is unsubstituted or substituted by halogen, cyano, C₁-C₄-alkoxy, C₁-C₄-alkylcarbonyl, C₁-C₄-alkoxycarbonyl, carbamoyl or di-C₁-C₄-alkylcarbamoyl, a C₃-C₆-cycloalkyl group, a C₃-C₆-alkenyl group, which is straight-chain or branched-chain, and is unsubstituted or substituted by halogen, a C₃-C₆-alkynyl group, which is straight-chain or branched-chain, and is unsubstituted or substituted by halogen,
a group

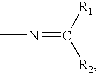

wherein R₁ and R₂ separately are each a C₁-C₄-alkyl group, or together form a 4- or 5-membered methylene chain, which can be substituted by C₁-C₄-alkyl; and/or

b) comprises an amount, which is effective for antagonism of a herbicide, of at least one quinoline derivative safener;

wherein the emulsifiable concentrate has a pH when diluted to 1% concentration in distilled water in the range of 4.5 to 8.0, preferably 5.0 to 7.0; and

wherein the emulsifiable concentrate contains less than 2.5%, preferably less than 2.0%, water.

It is also an object of the invention to provide selective pesticidal compositions obtainable by diluting the emulsifiable concentrates of the present invention in a suitable amount of water to form an oil-in-water emulsion.

It is another object of the invention to provide a method for the selective control of weeds in crops of useful plants, which method comprises treating the useful plants, their seeds or seedlings or the crop area thereof with the pesticidal compositions of the present invention.

Handling benefits of the present invention include excellent compatibility with fertilizers, low viscosity and low temperature stability, without crystallization or phase separation.

These and other objects and features of the invention will be more apparent from the detailed description set forth hereinbelow, and from the appended claims.

### DETAILED DESCRIPTION OF THE PREFERRED EMBODIMENTS

It has surprisingly been found that improved stable EC formulations containing one or more herbicidally active ingredients and at least one oil adjuvant can be produced using one or more water-immiscible solvents, an emulsifying surfactant system enabling an oil-in-water emulsion to be formed when the formulation is added to water and by controlling the pH and moisture content of the EC.

The present invention accordingly relates to an emulsifiable concentrate comprising

a) a herbicidally effective amount, preferably 1 to 30% by weight, of at least one herbicide;

b) optionally, an amount, which is effective for antagonism of a herbicide, of at least one safener;

c) 5 to 80%, preferably 25 to 70%, by weight of at least one oil adjuvant, wherein said oil adjuvant comprises an oil of vegetable or animal origin, or a mineral oil, alkyl esters thereof or mixtures of those oils and oil derivatives;

d) an amount of at least one water-immiscible solvent sufficient to keep the AI and safener in solution in the presence of the adjuvant, preferably 5 to 70% by weight, more preferably 25 to 60% by weight; and

e) an emulsifying surfactant system in an amount sufficient to form an oil-in-water emulsion when the formulation is added to water, preferably between 1 and 30% by weight;

with the proviso that a) comprises a herbicidally effective amount of at least one compound of formula I

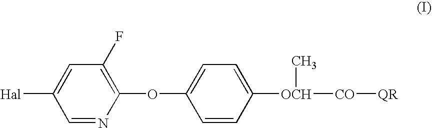

wherein Hal is halogen
Q is oxygen or sulfur,
R is hydrogen, an alkali metal ion, or a quaternary C₁-C₄-alkylammonium group, a C₁-C₆-alkyl group which is straight-chain or branched-chain, and which is unsubstituted or substituted by halogen, cyano, C₁-C₄-alkoxy, C₁-C₄-alkylcarbonyl, C₁-C₄-alkoxycarbonyl, carbamoyl or di-C₁-C₄-alkylcarbamoyl, a C₃-C₆-cycloalkyl group, a C₃-C₆-alkenyl group, which is straight-chain or branched-chain, and is unsubstituted or substituted by halogen, a C₃-C₆-alkynyl group, which is straight-chain or branched-chain, and is unsubstituted or substituted by halogen,
a group

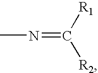

wherein R₁ and R₂ separately are each a C₁-C₄-alkyl group, or together form a 4- or 5-membered methylene chain, which can be substituted by C₁-C₄-alkyl; and/or

b) comprises an amount, which is effective for antagonism of a herbicide, of at least one quinoline derivative safener;

wherein the emulsifiable concentrate has a pH when diluted to 1% concentration in distilled water in the range of 4.5 to 8.0, preferably 5.0 to 7.0; and

wherein the emulsifiable concentrate contains less than 2.5%, preferably less than 2.0%, water.

Herbicides suitable for use in the emulsifiable concentrates of the present invention include PSII inhibitors, PSI inhibitors, ALS inhibitors, HPPD inhibitors, ACCase inhibitors, Cell Division inhibitors, PDS inhibitors, lipid metabolism inhibitors and PPGO inhibitors. Preferred classes of herbicides for use in the emulsifiable concentrates of the present invention comprise ACCase inhibitors and Cell Division inhibitors. Preferred herbicides include aryloxyphenoxypropionic herbicides including compounds of formula I, as described herein, clodinafop, cyhalofop, diclofop, fenoxaprop, fenoxaprop-P, fluazifop, haloxyfop, propaquizafop, quizalofop, quizalofop-P and agriculturally acceptable salts and esters thereof, such as, cyhalofop-butyl, diclofop-methyl, fenoxaprop-ethyl, fenoxaprop-P-ethyl, fluazifop-butyl, fluazifop-P-butyl, haloxyfop-etotyl, halyoxyfop-P-methyl, quizalofop-ethyl, quizalofop-P-ethyl and quizalofop-P-tefuryl; cyclohexanedione oxime herbicides including alloxydim, butroxydim, clethodim, cycloxydim, sethoxydim, tepraloxydim and tralkoxydim; acetamide herbicides such as diphenamid, napropamide, naproanilide, acetochlor, alachlor, butachlor, dimethachlor, dimethenamid, dimethenamid-P, fentrazamide metazachlor, metolachlor, pethoxamid, pretilachlor, propachlor, propisochlor, S-metolachlor, thenylchlor, flufenacet and mefenacet. As used herein, the term acetamide includes mixtures of the two or more acetamides as well as mixtures of optical isomers of the acetamides. For example, mixtures of the (R) and (S) isomers of metolachlor wherein the ratio of (S)-2-chloro-N-(2-ethyl-6-methylphenyl)-N-(2-methoxy-1-methylethyl)acetamide to (R)-2-chloro-N-(2-ethyl-6-methylphenyl)-N-(2-methoxy-1-methylethyl)acetamide is in the range of from 50-100% to 50-0%, preferably 70-100% to 30-0% and more preferably 80-100% to 20-0%; and pinoxaden.

These compounds are known in the art and are descnbed in The Pesticide Manual, Twelfth Edition, British Crop Protection Council or other readily available resources.

Particularly preferred herbicides comprise compounds of formula I wherein Hal is chlorine, Q is oxygen, and R is a C₃-C₆-alkynyl group, preferably a propynyl group. A preferred compound within the scope of formula I is commonly referred to as clodinafop-propargyl and is described, for example, in U.S. Pat. No. 4,713,109. The terms clodinafop and clodinafop-propargyl, as used herein, include mixtures of the (R) and (S) isomers of compounds of formula I wherein Hal is chlorine, Q is oxygen, and R is a propynyl group, and wherein the ratio of (R) to (S) is in the range of from 50-100% to 50-0%, preferably 80-100% to 20-0% and more preferably 90-100% to 10-0%.

In compounds of formula I the various substituents may contribute to optical isomerism and/or stereoisomerism. All such racemic mixtures and isomers are included within the scope of the present invention.

As used herein, the term “herbicidally effective amount” means the amount of herbicide compound which adversely controls or modifies plant growth.

The emulsifiable concentrates may contain an amount, which is effective for antagonism of a herbicide, i.e., an amount which counteracts to some degree a phytotoxic response of a beneficial crop to a herbicide, of at least one safener. Safeners suitable for use in the emulsifiable concentrates of the present invention include quinoline derivatives; benoxacor; dichlormid; fenchlorazole-ethyl; fenclorim; flurazole; fluxofenim; furilazole; isoxadifen-ethyl; mefenpyr; an alkali metal, alkaline earth metal, sulfonium or ammonium cation of mefenpyr; mefenpyr-diethyl and oxabetril.

Suitable quinoline derivatives for use as component b) of the present invention include those taught in U.S. Pat. Nos. 4,902,340 and 5,102,445, the contents of which are incorporated herein by reference. Preferred quinoline derivatives include cloquintocet; an alkali metal, alkaline earth metal, sulfonium or ammonium cation of cloquintocet; and cloquintocet-mexyl. A particularly preferred quinoline derivative is cloquintocet-mexyl.

In an embodiment of the present invention, clodinafop-propargyl is preferably used together with the safener cloquintocet-mexyl. These mixtures are described, for example, in U.S. Pat. No. 4,881,966. These mixtures are commercially available in the form of an emulsifiable concentrate.

The emulsifiable concentrates may further contain a weak acid or base that is soluble or fully miscible in the formulation and that has a pKa between about 1 and 8 in an amount sufficient to obtain an emulsifiable concentrate having a pH when diluted to 1% concentration in distilled water in the range of 4.5 to 8.0. Preferred acid components include organic acids, such as acetic acid, and acidic surfactants that can also function as an emulsifier.

Preferred oil adjuvants comprise mineral oils or an oil of vegetable origin such as, for example, canola oil, olive oil or sunflower oil, emulsified vegetable oil, alkyl esters of oils of vegetable origin such as, for example, the methyl derivatives, or an oil of animal origin such as fish oil or beef tallow.

Esters of plant oils are alkyl esters obtainable from medium chained fatty acids by esterification with alkanols or by transesterification of the corresponding plant oils. Preferred fatty acids of these plant oils have 5 to 20, in particular 8 to 18 carbon atoms. In a preferred embodiment the methyl ester of the plant oil used is the methyl ester of canola oil.

Especially preferred oil adjuvants comprise alkyl esters of higher fatty acids (C₈-C₂₂), especially the methyl derivatives of C₁₂-C₁₈ fatty acids, for example the methyl esters of lauric acid, palmitic acid and oleic acid. Those esters are known as methyl laurate (CAS-111-82-0), methyl palmitate (CAS-112-39-0) and methyl oleate (CAS-112-62-9).

The application and action of the oil adjuvants may be improved by combining them with surface-active substances such as non-ionic, anionic or cationic surfactants.

The emulsifying surfactant system enabling the EC to form an oil-in-water emulsion when the formulation is added to water is typically a mixture of two or more surfactants, at least one of which is a nonionic surfactant and optionally at least one of which is an anionic surfactant.

Suitable surface-active compounds are, depending on the nature of the active ingredient of formula I being formulated, non-ionic, cationic and/or anionic surfactants and mixtures of surfactants having good emulsifying, dispersing and wetting properties. Examples of suitable anionic, non-ionic and cationic surfactants are listed, for example, in U.S. Pat. No. 6,063,732 column 5, line 1 to column 6, line 2, the contents of which are incorporated herein by reference.

Furthermore, the surfactants customarily employed in formulation technology, which are described, inter alia, in “Mc Cutcheon's Detergents and Emulsifiers Annual” MC Publishing Corp., Ridgewood N.J., 1981, Stache, H., “Tensid-Taschenbuch”, Carl Hanser Verlag, MunichNienna, 1981 and M. and J. Ash, “Encyclopedia of Surfactants”, Vol I-III, Chemical Publishing Co., New York, 1980-81, are also suitable for preparation of the herbicidal compositions according to the invention.

Anionic surfactants suitable for use in the invention may be any known in the art. The anionic surfactants may be polyarylphenol polyalkoxyether sulfates and/or phosphates; C₈₋₁₈ alcohol polyalkoxyether phosphates, carboxylates, and/or citrates; alkyl benzenesulfonic acids; C₈₋₂₀ alkyl carboxylates including fatty acids; C₈₋₂₀ alcohol sulfates; C₈₋₂₀ alcohol phosphate mono- and diesters; C₈₋₂₀ alcohol and (C₈₋₂₀alkyl)phenol polyoxyethylene ether carboxylates, sulfates and sulfonates; C₈₋₂₀ alcohol and (C₈₋₂₀ alkyl)phenol polyoxyethylene phosphate mono- and diesters; C₈₋₂₀ alkylbenzene sulfonates, naphthalene sulfonates and formaldehyde condensates thereof; lignosulfonates; C₈₋₂₀ alkyl sulfosuccinates and sulfosuccinamates; C₈₋₂₀ acyl glutamates, sarcosinates, isethionates and taurates; water-soluble soaps and mixtures thereof.

Exemplary polyarylphenol polyalkoxyether sulfates and phosphates include polyarylphenol polyethoxyether sulfates and phosphates, polyarylphenol polypropoxyether sulfates and phosphates, polyarylphenol poly(ethoxy/propoxy)ether sulfates and phosphates, and salts thereof. The term “aryl” includes, for example, phenyl, tolyl, naphthyl, tetrahydronaphthyl, indanyl, indenyl, styryl, pyridyl, quinolinyl, and mixtures thereof. Exemplary polyarylphenol polyethoxyether sulfates and phosphates include distyrylphenol polyethoxyether sulfates and phosphates, and tristyrylphenol polyethoxyether sulfates and phosphates. The polyarylphenol polyalkoxether sulfates and phosphates may have a degree of alkoxylation (e.g., ethoxylation) of between about 1 and about 50, preferably between about 2 and about 40, more preferably between about 5 and about 30. Commercially available polyarylphenol polyalkoxyether sulfates and phosphates include, for example, SOPROPHOR® 4 D 384 (Rhodia Corporation, Cranbury, N.J.) (tristyrylphenol (EO)₁₆ sulfate ammonium salt), SOPROPHOR® 3 D 33 (Rhodia Corporation, Cranbury, N.J.) (tristyrylphenol (EO)₁₆ phosphate free acid), SOPROPHOR® FLK (Rhodia Corporation, Cranbury, N.J.) (tristyrylphenol (EO)₁₆ phosphate potassium salt) and SOPROPHOR® RAM/384 (Rhodia Corporation, Cranbury, N.J.) (tristyrylphenol polyethoxylated ether sulfate neutralized with polyethoxylated oleylamine). In other embodiments, the polyarylphenol polyalkoxyether sulfates and phosphates may be mono-arylphenol polyalkoxyether sulfates and phosphates, such as styrylphenol polyethoxyether sulfates and phosphates.

Exemplary C₈₋₁₈ alcohol polyethoxyether phosphates, carboxylates and citrates include STEPFAC® 8180 (Stepan Corporation, Northfield, Ill.) (tridecylalcohol (EO)₃ phosphate), STEPFAC® 8181 (Stepan Corporation, Northfield, Ill.) (tridecylalcohol (EO)₆ phosphate), STEPFAC® 8182 (Stepan Corporation, Northfield, Ill.) (tridecylalcohol (EO)₁₂ phosphate), EMCOL® CN-6 (CK Witco Corporation, Greenwich, Conn.) (tridecylalcohol (EO)₆ carboxylate). The C₈₋₁₈ alcohol polyethoxyether phosphates, carboxylates and citrates may have a degree of ethoxylation of between about 1 and about 25, preferably between about 1 and about 20.

Exemplary alkylbenzene sulfonic acids and salts thereof include dodecylbenzene sulfonic acid, and metal (for example sodium or calcium), ammonia or amine salts of the alkylbenzene sulfonic acids, including dodecylbenzene sulfonic acid. Amine neutralized versions include primary amines, diamines, triamines and alkanol amines.

Additional preferred anionic surfactants include (C₈₋₁₂ alkyl)phenol polyoxyethylene ether sulfates, and (C₈₋₁₂ alkyl)phenol polyoxyethylene phosphate mono- and diesters, accompanied in each case by monovalent counterions. In one embodiment the monovalent counterion for a (C₈₋₁₂ alklyl)phenol polyoxyethylene ether sulfate or a (C₈₋₁₂ alkyl)phenol polyoxyethylene phosphate is a protonated polyoxyethylene C₁₂₋₂₀ alkylamine surfactant. More specifically, polyoxyethylene tallowamine salt of a nonylphenol polyoxyethylene ether sulfate, nonylphenol polyoxyethylene phosphate, and a blend of such nonylphenol polyoxyethylene phosphate with polyoxyethylene tallowamine.

Suitable water-soluble soaps are the alkali metal salts, alkaline earth metal salts, ammonium salts or substituted ammonium salts of higher fatty acids (C_{10-C}₂₂), e.g. the sodium or potassium salts of oleic or stearic acid, or of natural fatty acid mixtures which can be obtained, inter alia, from coconut oil or tallow oil. Further suitable soaps are also the fatty acid methyl taurin salts.

The anionic surfactants are optionally neutralized with a basic compound. The basic compounds may be any known in the art that are capable of neutralizing the anionic surfactants. Basic compounds include, for example, inorganic bases, C₈₋₁₈ alkyl amine polyalkoxylates, alkanol amines, alkanol amides, and mixtures thereof.

Exemplary inorganic bases include ammonium hydroxides, sodium hydroxides, potassium hydroxides, calcium hydroxides, magnesium hydroxides, zinc hydroxides, and mixtures thereof. The C₈₋₁₈ alkyl amine polyalkoxylates may be, for example, C₈₋₁₈ alkyl amine polypropoxylates and/or C₈₋₁₈ alkyl amine polyethoxylates. Exemplary C₈₋₁₈ alkyl amine polyalkoxylates include tallow amine polyalkoxylates, cocoamine polyalkoxylates, oleylamine polyalkoxylates, and stearylamine polyalkoxylates. The C₈₋₁₈ alkyl amine polyethoxyates may have from about 2 to about 50 moles of ethylene oxide per molecule, more preferably from about 2 to about 20 moles of ethylene oxide per molecule. Exemplary C₈₋₁₈ alkyl amine polyethoxylates include tallow amine ethoxylates (2 moles EO or 8 moles EO), cocoamine ethoxylates, oleylamine ethoxylates, and stearylamine ethoxylates. Exemplary alkanol amines include diethanol amine and triethanol amine. Exemplary alkanol amides include oleic diethanolamide and linoleic diethanolamide, and the diethanolamides of other C₈₋₁₈ fatty acids.

The anionic surfactants may be neutralized to the inflection point in the titration curve with one or more basic compounds. One skilled in the art will recognize that the pH of the inflection will vary according to the acid and base strengths of the components being used, but will generally fall within the range of about pH 4 to about pH 9, preferably about pH 5 to about pH 7. For example, the compositions of the invention may comprise at least one polyarylphenol polyalkoxyether sulfate, polyarylphenol polyalkoxyether phosphate, C₈₋₁₈ alcohol polyalkoxyether phosphates, C₈₋₁₈ alcohol polyalkoxyether carboxylates, C₈₋₁₈ alcohol polyalkoxyether citrates, and/or alkyl benzenesulfonic acids neutralized to the inflection point in the titration curve with one or more basic compounds. The basic compound used to neutralize the different anionic surfactants may be the same or different.

In still other embodiments, the compositions of the invention comprise mixtures of at least two anionic surfactants selected from polyarylphenol polyalkoxyether sulfates, polyarylphenol polyalkoxyether phosphates, C₈₋₂₀ alkyl carboxylates including fatty acids, C₈₋₂₀ alcohol sulfates, C₈₋₂₀ alcohol phosphate mono- and diesters, C₈₋₂₀ alcohol and (C₈₋₂₀ alkyl)phenol polyoxyethylene ether carboxylates, sulfates and sulfonates, C₈₋₂₀ alcohol and (C₈₋₂₀ alkyl)phenol polyoxyethylene phosphate mono- and diesters, C₈₋₂₀ alkylbenzene sulfonates, naphthalene sulfonates and formaldehyde condensates thereof, lignosulfonates, C₈₋₂₀ alkyl sulfosuccinates and sulfosuccinamates, and/or C₈₋₂₀ acyl glutamates, sarcosinates, isethionates and taurates neutralized to the inflection point in the titration curve with one or more basic compounds. The basic compound used to neutralize the different anionic surfactants may be the same or different.

When neutralized, the anionic surfactants and basic compounds are preferably used in a ratio of about 1:1. One basic compound may be used to neutralize one or more anionic surfactants. In other embodiments, more than one basic compound may be used to neutralize one or more anionic surfactants.

Exemplary nonionic surfactants include ethylene oxide-propylene oxide block copolymers; ethylene oxide-butylene oxide block copolymers; C₂₋₆ alkyl adducts of ethylene oxide-propylene oxide block copolymers; C₂₋₆ alkyl adducts of ethylene oxide-butylene oxide block copolymers; polypropylene glycols; polyethylene glycols; polyarylphenol polyethoxy ethers; polyalkylphenol polyethoxy ethers; polyglycol ether derivatives of aliphatic or cycloaliphatic alcohols or of saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols; mono-, di- and tri(C₁₂₋₂₀ alkyl)esters of polyoxyethylene sorbitan; alkoxylated vegetable oils; alkoxylated acetylenic diols; alkyl polyglycosides and mixtures thereof.

The ethylene oxide-propylene oxide block copolymers may comprise alkyl or alkyphenol ether bases, such as butyl ether, methyl ether, propyl ether, ethyl ether, or mixtures thereof. Commercially available nonionic surfactants include, for example, TOXIMUL® 8320 (Stepan Corporation, Northfield, Ill.) (butyl ether derivative of EO/PO block copolymer), WITCONOL® NS-500LQ (CK Witco Corporation, Greenwich, Conn.) (butyl ether derivative of EO/PO block copolymer) and WITCONOL® NS-108LQ (CK Witco Corporation, Greenwich, Conn.) (nonylphenol ether derivative of EO/PO block copolymer).

Other suitable non-ionic surfactants are the water-soluble, 20 to 250 ethylene glycol ether groups containing polyadducts of ethylene oxide and propylene oxide, ethylene diamino polypropylene glycol and alkyl polypropylene glycol with 1 to 10 carbon atoms in the alkyl moiety, the substances normally contain 1 to 5 ethylene glycol units per propylene glycol unit. Examples of non-ionic surfactants are nonylphenol polyethoxy ethanols, vegetable oil polyglycol ethers, polyadducts of ethylene oxide and propylene oxide, tributyl phenoxy polyethoxy ethanol, octyl phenoxy polyethoxy ethanol. Preferred are fatty acid esters of polyoxy ethylene sorbitan, such as polyoxy ethylene sorbitan trioleate.

Preferably, the surfactant system according to the present invention consists of an anionic surfactant and one or two non-ionic surfactants.

Cationic surfactants are preferably quaternary ammonium salts carrying, as N-substituent, at least one C₈-C₂₂ alkyl radical and, as further substituents, unsubstituted or halogenated lower alkyl, benzyl or hydroxy-lower alkyl radicals. The salts are preferably in the form of halides, methyl sulfates or ethyl sulfates, for example stearyl trimethylammonium chloride or benzyl bis(2-chloroethyl)ethylammonium bromide.

The amount of surfactant(s) depends on the particular active ingredients selected for the composition and the absolute and relative amounts of these desired. Suitable amounts of stabilizing system components selected from the classes or specific examples provided herein can be determined by routine experimentation, the test being that substantially no phase separation, sedimentation or flocculation is exhibited by the composition following storage at 20-25° C. for a period of 24 hours, or, for preferred embodiments, following a longer period of storage over a broader range of temperatures as indicated above. Typically the total concentration of all surfactants in the composition as a whole is about 1% to about 30% by weight, excluding the weight of counterions, if present.

In computing relative amounts of surfactants present in a composition, the weight of water or other diluent supplied with a surfactant, if known, should be excluded. For example, WITCONATE® 79S of CK Witco Corporation contains 52% dodecylbenzene sulfonic acid triethanolamine salt. In a composition containing 1% WITCONATE® 79S, the concentration of dodecylbenzene sulfonic acid triethanolamine salt should be computed as 0.52%.

Suitable water-immiscible solvents are preferably selected from the group consisting of aromatic hydrocarbons, aliphatic hydrocarbons, dialkylene glycol dialkyl ethers, ketones, alkyl esters of acetic acid or mixtures thereof.

Aromatic and aliphatic hydrocarbons such as hexane, cyclohexane, benzene, toluene, xylene, mineral oil or kerosene or substituted naphthalenes, mixtures of mono- and polyalkylated aromatics are commercially available under the registered trademarks Solvesso® and Shellsol® and Petrol Spezial®. Preferred ketones include acetophenone. Representative alkyl esters of acetic acid include EXXATE™ fluids, such as EXXATE™ 800, available from ExxonMobil Chemical Company, Houston, Tex.

A safener in accordance with the invention may, depending on the intended purpose, be used to pre-treat the seed material of the cultivated plant (dressing the seed or the seedlings) or may be incorporated into the soil before or after sowing. It may, however, also be applied, alone or together with the herbicide and the oil adjuvant, after the emergence of the plants. The treatment of the plants or seed with the safener can therefore, in principle, be effected independently of the time at which the herbicide is applied. The treatment of the plants can, however, also be carried out by applying the herbicide, oil adjuvant and safener simultaneously. The rate of application of the safener in relation to the herbicide depends largely on the method of application. In the case of field treatment, which is effected either using a tank mixture with a combination of the safener and the herbicide or by the separate application of the safener and the herbicide, the ratio of herbicide to safener is generally from 100:1 to 1:10, preferably from 20:1 to 1:1. In the case of field treatment, from 0.001 to 1.0 kg of safener/ha, preferably from 0.001 to 0.25 kg of safener/ha, are generally applied.

It is also an object of the invention to provide selective pesticidal compositions obtainable by diluting the emulsifiable concentrate of the present invention in a suitable amount of water to form an oil-in-water emulsion.

It is another object of the invention to provide a method for the selective control of weeds in crops of useful plants, which method comprises treating the useful plants, their seeds or seedlings or the crop area thereof with the pesticidal compositions of the present invention.

The ECs of the present invention may preferably be in a concentrated form whereas the end-user generally employs diluted compositions. Said compositions may be diluted to concentrations down to 0.01% of active ingredient (a.i.). The compositions are typically applied in amounts sufficient to deliver from about 0.006 to 0.6 kg a.i./ha.

The composition according to the invention is suitable for all methods of application conventionally used in agriculture, e.g. pre-emergence application, post-emergence application and seed dressing. The compositions according to the invention are preferably used for post-emergence control of weeds.

The composition according to the invention is suitable especially for controlling weeds in crops of useful plants, such as cereals, rape, sugar beet, sugar cane, plantation crops, rice, maize and soybeans. Particularly preferred crops are maize, rice, soybeans and cereals such as wheat or barley. “Crops” are to be understood also to include those crops that have been made tolerant to herbicides or classes of herbicides as a result of conventional methods of breeding or genetic engineering. The components used in the composition of the invention can be applied in a variety of ways known to those skilled in the art, at various concentrations. The rate at which the herbicidal compositions are applied will depend upon the particular type of weed to be controlled, the degree of control required, and the timing and method of application.

Crop areas are areas of land on which the cultivated plants are already growing or in which the seeds of those cultivated plants have been sown, and also areas of land on which it is intended to grow those cultivated plants.

The weeds to be controlled may be either dicotyledonous or, preferably, monocotyledonous weeds, for example the monocotyledonous weeds Avena, Agrostis, Phalaris, Lolium, Bromus, Alopecurus, Setaria, Digitaria, Brachiaria, Echinochloa, Panicum, Sorghum hal./bic., Rottboellia, Cyperus, Brachiaria, Echinochloa, Scirpus, Monochoria and Sagittaria and the dicotyledonous weeds Sinapis, Chenopodium, Stellaria, Galium, Viola, Veronica, Matricaria, Papaver, Solanum, Abutilon, Sida, Xanthium, Amaranthus, Ipomoea and Chrysanthemum.

Other active ingredients such as co-herbicides, fungicides, insecticides, acaricides and nematicides may be present in the emulsifiable concentrate or may be added as a tank-mix partner with the emulsifiable concentrate.

These compositions may also comprise other auxiliaries such as chemical stabilizers, viscosity controlling agents, thickeners, binders, tackifiers, fertilizers and anti-foam agents.

Suitable stabilizers include epoxidized animal or vegetable oils, such as epoxidized soybean oil.

Representative anti-foam agents are silica, polydialkylsiloxanes, in particular polydimethylsiloxanes, fluoroaliphatic esters or perfluoroalkylphosphonic/perfluoroalkylphosphonic acids or the salts thereof and mixtures thereof. Preferred are polydimethylsiloxanes.

For a clearer understanding of the invention, specific examples are set forth below. These examples are merely illustrations and are not to be understood as limiting the scope and underlying principles of the invention in any way. Indeed, various modifications of the invention in addition to those shown and described herein will become apparent to those skilled in the art from the following examples and foregoing description.

### Preparation of Test Samples

In order to demonstrate the effect of water on the stability of the compositions of the present invention, a series of emulsifiable concentrate formulations were prepared by dissolving clodinifop-propargyl, cloquintocet-mexyl, Atplus® 300F surfactant blend and water in a mixture of methylated rape seed oil and Exxate 800 in the proportions (wt %) given in Table 1 below. The pH of the compositions when diluted to 1% concentration in water was measured by pH electrode.

Samples of the emulsifiable concentrate compositions were placed in storage at a temperature of 70° C., and the concentration of clodinofop-propargyl and cloquintocet-mexyl ingredients was measured over time by gas chromatographic techniques known in the art. Based on these measurements a half-life for each ingredient could be calculated.

TABLE 1

| Composition | 1 | 2 | 3 | 4 |
|---|---|---|---|---|
| Clodinofop-propargyl | 6.5 | 6.5 | 6.5 | 6.5 |
| Cloquintocet-mexyl | 1.7 | 1.7 | 1.7 | 1.7 |
| Atplus 300 F Surfactant | 13.2 | 13.2 | 13.2 | 13.2 |
| Methylated Rape Seed Oil | 34.8 | 34.8 | 34.8 | 34.8 |
| Exxate 800 | 40.8 | 42.8 | 43.5 | 43.7 |
| Water | 3.0 | 1.0 | 0.3 | 0.1 |
| Total | 100.0 | 100.0 | 100.0 | 100.0 |
| pH (1% solution) | 5.61 | 5.67 | 5.78 | 5.51 |
| Clodinofop-p Half-Life | 39.8 | 50.4 | 54.4 | 58.2 |
| at 70° C. (Days) | | | | |
| Cloquintocet-m Half-Life | 18.7 | 23.4 | 25.3 | 26.3 |
| at 70° C. (Days) | | | | |

The half-life data of Table 1 indicate that the water content of the compositions strongly influence the chemical stability of the active ingredients during storage. The optimum chemical stability of the active ingredients is observed when the water content is maintained below about 3%.

In order to demonstrate the effects of pH on the stability of the compositions of the present invention, emulsifiable concentrate formulations were prepared by dissolving clodinifop-propargyl, cloquintocet-mexyl, Atplus® 300F, AgRHOSpec 6713, Bio-Soft S-101 and water in a mixture of methylated rape seed oil, acetophenone and Exxate 800 in the proportions (wt %) given in Table 2 below. The compositions exhibit a range of pH values when diluted to 1% concentration in water.

TABLE 2

| Composition | 5 | 6 | 7 | 8 | 9 | 10 |
|---|---|---|---|---|---|---|
| Clodinofop- | 6.5 | 6.5 | 6.5 | 6.5 | 6.5 | 6.5 |
| propargyl | | | | | | |
| Cloquintocet- | 1.7 | 1.7 | 1.7 | 1.7 | 1.7 | 1.7 |
| mexyl | | | | | | |
| Atplus 300 | 13.2 | 13.2 | 13.2 | 13.2 | 13.2 | 13.2 |
| F Surfactant | | | | | | |
| Methylated | 34.9 | 34.9 | 34.9 | 34.9 | 34.9 | 34.9 |
| Rape Seed Oil | | | | | | |
| Acetophenone | 7.0 | 7.0 | 7.0 | 7.0 | 7.0 | 7.0 |
| Exxate 800 | 30.07 | 30.18 | 30.08 | 30.11 | 30.08 | 30.07 |
| Water | 1.45 | 1.43 | 1.44 | 1.41 | 1.44 | 1.45 |
| AgRHOSpec | 4.36 | 4.45 | 4.49 | 4.49 | 4.52 | 4.66 |
| 6713 | | | | | | |
| Bio-Soft S-101 | 0.82 | 0.73 | 0.69 | 0.69 | 0.66 | 0.52 |
| Total | 100.00 | 100.00 | 100.00 | 100.00 | 100.00 | 100.00 |
| pH (1% | 4.39 | 4.91 | 5.15 | 5.50 | 5.72 | 7.76 |
| solution) | | | | | | |
| Clodinofop-p | 28.1 | 48.3 | 59.0 | 65.1 | 69.0 | 47.6 |
| Half-Life at | | | | | | |
| 70° C. (Days) | | | | | | |
| Cloquintocet-m | 18.6 | 17.9 | 21.3 | 27.0 | 33.9 | 36.7 |
| Half-Life at | | | | | | |
| 70° C. (Days) | | | | | | |

Samples of the emulsifiable concentrate compositions were placed in storage at a temperature of 70° C., and the concentration of clodinofop-propargyl and cloquintocet-mexyl ingredients was measured over time by gas chromatographic techniques known in the art. Based on these measurements a half-life for each ingredient could be calculated.

The data of Table 2 indicate that the pH of the compositions strongly influence the chemical stability of the active ingredients during storage. The optimum chemical stability of the active ingredients is observed when the pH is maintained between about 4.5 and 8.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

We claim:

1. An emulsifiable concentrate comprising:
a) a herbicidally effective amount, preferably 1 to 30% by weight, of at least one herbicide;
b) optionally, an amount, which is effective for antagonism of a herbicide, of at least one safener;
c) 5 to 80%, preferably 25 to 70%, by weight of at least one oil adjuvant, wherein said oil adjuvant comprises an oil of vegetable or animal origin, or a mineral oil, alkyl esters thereof or mixtures of those oils and oil derivatives;
d) an amount of at least one water-immiscible solvent sufficient to keep the AI and safener in solution in the presence of the adjuvant, preferably 5 to 70% by weight, more preferably 25 to 60% by weight; and
e) an emulsifying surfactant system in an amount sufficient to form an oil-in-water emulsion when the formulation is added to water, preferably between 1 and 30% by weight;
with the proviso that a) comprises a herbicidally effective amount of at least one compound of formula I 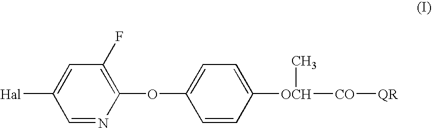
wherein Hal is halogen
Q is oxygen or sulfur,
R is hydrogen, an alkali metal ion, or a quaternary C₁-C₄-alkylammonium group, a C₁-C₆-alkyl group which is straight-chain or branched-chain, and which is unsubstituted or substituted by halogen, cyano, C₁-C₄-alkoxy, C₁-C₄-alkylcarbonyl, C₁-C₄-alkoxycarbonyl, carbamoyl or di-C₁-C₄-alkylcarbamoyl, a C₃-C₆-cycloalkyl group, a C₃-C₆-alkenyl group, which is straight-chain or branched-chain, and is unsubstituted or substituted by halogen, a C₃-C₆-alkynyl group, which is straight-chain or branched-chain, and is unsubstituted or substituted by halogen,
a group 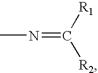
wherein R₁ and R₂ separately are each a C₁-C₄-alkyl group, or together form a 4- or 5-membered methylene chain, which can be substituted by C₁-C₄-alkyl; and/or
b) comprises an amount, which is effective for antagonism of a herbicide, of at least one quinoline derivative safener;
wherein the emulsifiable concentrate has a pH when diluted to 1% concentration in distilled water in the range of 4.5 to 8.0, preferably 5.0 to 7.0; and
wherein the emulsifiable concentrate contains less than 2.5%, preferably less than 2.0%, water.

2. The emulsifiable concentrate of claim 1 comprising a herbicidally effective amount of a compound of formula I.

3. The emulsifiable concentrate of claim 2 wherein the compound of formula I is clodinafop-propargyl.

4. The emulsifiable concentrate of claim 1 wherein the oil adjuvant comprises a methyl ester of a plant oil.

5. The emulsifiable concentrate of claim 3 wherein the oil adjuvant comprises a methyl ester of canola oil.

6. The emulsifiable concentrate of claim 1 wherein the emulsifying surfactant system comprises at least one non-ionic surfactant.

7. The emulsifiable concentrate of claim 1 comprising b) at least one safener selected from the group consisting of quinoline derivatives; benoxacor; dichlormid; fenchlorazole-ethyl; fenclorim; flurazole; fluxofenim; furilazole; isoxadifen-ethyl; mefenpyr, an alkali metal, alkaline earth metal, sulfonium or ammonium cation of mefenpyr, mefenpyr-diethyl and oxabetrinil.

8. The emulsifiable concentrate of claim 7 wherein the safener comprises a quinoline derivative.

9. The emulsifiable concentrate of claim 8 wherein the quinoline derivative comprises at least one member selected from the group consisting of cloquintocet; an alkali metal, alkaline earth metal, sulfonium or ammonium cation of cloquintocet; and cloquintocet-mexyl.

10. The emulsifiable concentrate of claim 9 wherein the safener comprises cloquintocet-mexyl.

11. The emulsifiable concentrate of claim 1 wherein the herbicide comprises cloquintocet-mexyl and the safener comprises cloquintocet-mexyl.

12. The emulsifiable concentrate of claim 1 wherein the pH is in the range of from 5.0 to 7.0.

13. The emulsifiable concentrate of claim 1 wherein the water content is less than 2.0% by weight.

14. The emulsifiable concentrate of claim 1 further comprising at least one member selected from the group consisting of co-herbicides, fungicides, insecticides, acaricides and nematicides.

15. The emulsifiable concentrate of claim 1 further comprising at least one member selected from the group consisting of chemical stabilizers, viscosity controlling agents, thickeners, binders, tackifiers, fertilizers and anti-foam agents

16. A pesticidal composition obtained by diluting an emulsifiable concentrate according to claim 1 in a suitable amount of water to form an oil-in-water emulsion.

17. The pesticidal composition of claim 16 further comprising at least one member selected from the group consisting of co-herbicides, fungicides, insecticides, acaricides, and nematicides

18. A method for the selective control of weeds in crops of useful plants, which method comprises treating the useful plants, their seeds or seedlings or the crop area thereof with a pesticidal composition according to claim 16.

19. The method according to claim 18 wherein the crops of useful plants are selected from the group consisting of maize, cereals, rice and soybeans.

20. The method according to claim 19 wherein the crops of useful plants are cereals.

21. The method according to claim 20 wherein the crop of useful plants is wheat or barley.
